(19) United States
(12) Patent Application Publication
(10) Pub. No.: US 2008/0045877 A1
(43) Pub. Date: Feb. 21, 2008

(54) BLOOD EXCHANGE DIALYSIS METHOD AND APPARATUS

(75) Inventors: Howard Levin, Teaneck, NJ (US); Mark Gelfand, New York, NY (US)

Correspondence Address: NIXON & VANDERHYE, PC, 901 NORTH GLEBE ROAD, 11TH FLOOR, ARLINGTON, VA, 22203, US

(73) Assignee: G&L Consulting, LLC, New York, NY (US)

(21) Appl. No.: 11/839,305

(22) Filed: Aug. 15, 2007

Related U.S. Application Data
(60) Provisional application No. 60/822,423, filed on Aug. 15, 2006.

(51) Int. Cl. A61M 1/36

(52) U.S. Cl. 604/4.01

(57) ABSTRACT

A method and apparatus for replacement of renal function by periodically removing a substantial volume of patient's blood and simultaneously replacing it with the reconstituted blood. Reconstituted blood consists of the patient's own condensed blood cells and proteins diluted with the sterile physiologic solution. As a result, small molecules and excess water are periodically removed and discarded. Blood cells and proteins are safely stored to be used at the next therapy session.

### RELATED APPLICATION

This application claims the benefit of U.S. Provisional Application Ser. No. 60/822,423 (NV 4343-35) filed Aug. 15, 2006, the entirety of which provisional application is incorporated by reference herein.

### BACKGROUND OF THE INVENTION

This invention relates to methods and apparatus for treatment of End Stage Renal Disease (ESRD) with Renal Replacement Therapy (RRT), artificial kidney and blood dialysis. It also relates to blood transfusion and separation of blood components.

### End Stage Renal Disease (ESRD):

A healthy human kidney continuously removes waste products (solute) and excess water from the blood. ESRD is a slow, progressive loss of kidney function caused by inherited disorders, prolonged medical conditions such as diabetes and hypertension or the long-term use of certain medications. ESRD is irreversible and lethal if untreated. Life can be sustained only through transplantation or dialysis (typically hemodialysis or blood dialysis). Transplantation is severely limited due to the shortage of suitable donors, the incidence of organ transplant rejection and the age and health of many ESRD patients. The vast majority of patients, therefore, must rely on dialysis for the remainder of their lives. Based upon information published by the U.S. Government, the approximate number of ESRD patients in the United States requiring dialysis treatments has grown from 66,000 at the end of 1982 to 260,000 at the end of 2000, representing a compound annual growth rate of approximately 8%. In addition, according to international patient registries, there were approximately 450,000 dialysis patients in Europe and Japan in 1999. Annual cost of maintenance dialysis in the United States is estimated to have exceeded $5 billion. The total world need for dialysis is much greater and largely unfulfilled in less affluent and developing countries.

### Existing Methods of Renal Replacement Therapy:

Renal Replacement Therapy (RRT) performs two primary functions: ultrafiltration (removal of water from blood plasma), and solute clearance (removal of different molecular weight substances from blood plasma). Dialysis is the dominating modality of RRT today. The filter called a “dialyzer” can be set up to perform either one or both of these functions simultaneously, with or without fluid replacement, accounting for the various modes of renal replacement therapy. “Clearance” is the term used to describe the removal of solute substances, both normal and waste product, from the blood. The toxic substances that need to be removed by RRT are dissolved in the plasma water.

In medicine, term dialysis or hemodialysis, designates a method for removing waste products such as potassium and urea, as well as free water from the blood when the kidneys are incapable of this (i.e. in renal failure). It is a form of RRT. Maintenance dialysis is needed for patients with ESRD who have none or very little natural residual renal function left. Hemodialysis is typically conducted in a dedicated facility, either a special room in a hospital or a clinic (with specialized nurses and technicians) that specializes in hemodialysis. Although less typical (especially in the USA), dialysis can also be done in a patient's home. Home hemodialysis has numerous advantages over in-center dialysis, such as greater patient control over the therapy and better symptom control due to longer and/or more frequent dialysis sessions. It is used infrequently because of the complexity of equipment and associated risks.

Hemodialysis usually also involves the removal (ultrafiltration) of extra fluid from the patient, because most patients with end-stage renal failure pass no urine. The sudden removal of fluid on dialysis may cause side effects, which are usually proportionate to the amount of fluid which is removed. These potential side effects include low blood pressure, fatigue, chest pains, leg-cramps and headaches. It is logical that more frequent “net” removal of less fluid should result in fewer side effects. Ultrafiltration is the convective transfer of fluid out of the plasma compartment through pores in the membrane. The pores filter electrolytes and small and middle sized molecules (up to 20,000 to 30,000 daltons) from the blood plasma. The ultrafiltrate output from the filtration pores is similar to plasma, but without the plasma proteins or cellular components. Importantly, since the concentration of small solutes is the same in the ultrafiltrate as in the plasma, no clearance is obtained, but fluid volume is removed.

Dialysis (hemodialysis) is the diffusive transfer of small solutes out of a blood plasma compartment by diffusion across the membrane itself. It occurs as a result of a concentration gradient, with diffusion occurring from the compartment with higher concentration (typically the blood compartment) to the compartment with lower concentration (typically the dialysate compartment). Since the concentration of solutes in the plasma decreases, clearance is obtained, but fluid may not be removed. However, ultrafiltration can be combined with dialysis.

Hemofiltration is the combination of ultrafiltration, and fluid replacement typically in much larger volumes than needed for fluid control. The replacement fluid contains the electrolytes desired to remain in the patient's blood, but not other, undesired small molecules. Since the net effect of replacing fluid without small solutes and ultrafiltration of fluid with small solutes results in net removal of small solutes, clearance is obtained.

### Limitations of Existing RRT:

Substantially unchanged over last 30 years, outpatient hemodialysis has produced relatively poor clinical outcomes, high total treatment costs and low quality of life for dialysis patients. These clinical outcomes are reflected in the mortality rates of dialysis patients. Mortality in patients is highly correlated to the dose of dialysis delivered to patients. In general, the dose of dialysis depends on the performance of the artificial kidney, patient size and the duration of treatment. Patients receiving outpatient hemodialysis experience a number of chronic and acute health problems. The chronic problems include hypertension, anemia (low red blood cell count), malnutrition, fluid and electrolyte imbalance, calcium deficiency, insomnia, sexual impotency, decreased mental acuity and lower energy levels. The acute problems include headaches, nausea, hypotension and asthenia (a general lack of strength and vitality), which are associated with currently standard in the U.S. three times per week dialysis sessions. In addition, a general feeling of ill health tends to increase between dialysis treatments as a result of toxins, sodium and water building up in the patient's blood.

It is believed that these health problems are caused in large part by inadequate dose of dialysis. The amount of toxins removed from the blood during dialysis is widely accepted to be determined by a formula indicating that hemodialysis is most efficient in the earlier stages (typically first 2 hours) of therapy. The reduction of efficiency of dialysis in removing toxins over time is explained by the time needed for solute to diffuse from different body compartments into blood. Thus, simply increasing the duration of a treatment session is not an efficient way to improve the dose of hemodialysis. Rather, the efficiency of hemodialysis and the delivered dose can be improved with more frequent dialysis sessions of shorter duration. Further, reports have noted that more frequent sessions also decrease the severe oscillations in toxin and hydration levels associated with the prevailing thrice weekly dialysis regimen and as a consequence, result in fewer side effects. Despite the potential benefits of frequent (such as daily) hemodialysis, several barriers have prevented it from becoming a viable treatment regimen. The most significant is the economic implication of administering hemodialysis to patients every day. Dialysis providers cannot afford the additional costs that would be incurred by providing daily treatments in outpatient facilities. Requiring daily visits to a dialysis treatment facility, at 3-4 hours each visit, would also place additional burdens on a patient's lifestyle.

The clinical outcomes of conventional dialysis have contributed to the significant patient treatment cost. Total treatment costs per dialysis patient paid by Medicare in U.S. were $33,400 in 1988. Based on industry data, the cost per dialysis patient paid by Medicare in 1999 was approximately $68,400. The cost of hospitalization on a fee for service basis represents the most significant component of this increase. While reimbursement for outpatient hemodialysis treatment (the “composite rate”) has been capped since 1983, reimbursement for the associated cost of care due to chronic and acute health problems and other complications continues to be reimbursed on a fee for service basis. Under this reimbursement scheme, providers are forced to reduce the cost of outpatient dialysis rather than the total cost of treating dialysis patients.

For purposes of comparing the traditional dialysis to the present invention, some main limitations of traditional maintenance dialysis include among others:

i. Dialysis requires extracorporeal blood flow circulation through the filter of 300-400 ml/min for 4 hours. This necessitates surgically implanted Arteriovenous (AV) shunt grafts and AV shunt fistulas for blood access. These traditional methods for dialysis blood access are invasive, time consuming and prone to frequent infection, occlusion and clotting. Annual costs associated with simply keeping a viable access site in dialysis patients are estimated in excess of $7.5 billion worldwide.

ii. Traditional dialysis schedule is 4 to 5 hours at each of 3 dialysis sessions per week. Replacement of normal continuous renal function with infrequent sessions results in accumulation of fluid and toxins during interdialitic periods and poor quality of life for patients.

iii. During dialysis sessions patients need to be periodically monitored for hypo- and hypertension. Associated personnel cost further increases the overall cost of therapy.

iv. Traditional dialysis requires hundreds of liters of purified water for each session. Water purification (by reverse osmosis) is an energy consuming, technologically demanding process. The associated infrastructure increases the cost for dialysis providers. Need for large amount of sterile and purified water is considered the main obstacle to the adoption of home dialysis that is proven to be more beneficial to patients and society than periodic treatment center dialysis.

v. Dialysis is inefficient in removal of middle size molecules (3,000-12,000 Daltons) that diffuse less efficiently than small solutes across the filter membrane. This results in poor removal of some blood borne toxins leading to such complications as amylodosis, carpal tunnel syndrome and dysfunction of other vital organs.

With the increasing prevalence of ESRD, intractably poor clinical outcomes and quality of life of patients, and the increased cost of maintenance dialysis, a strong need has emerged for a new technology that will allow more frequent RRT sessions providing superior overall clinical benefit at lower cost. In addition, the development of dialysis methods with reduce or eliminate the need for maintenance of high blood flow dialysis access AV shunts is a separate goal to minimize the high cost and morbidity associated with this major clinical problem.

### Exchange Transfusion and Rapid Blood Exchange

Exchange transfusion is a well known potentially life-saving procedure performed to counteract the effects of serious jaundice or changes in the blood (from, for example, sickle cell anemia). The procedure involves the incremental removal of the patient's blood and replacement with fresh donor blood or plasma. In some patients, whole blood can be removed from one arm at the same time that donor cells are transfused into the other arm. In adults, this procedure can be performed in 500 mL units. The total volume of blood to be used is proportional to the patient's body weight and hematocrit; thus, different formulas are needed for different initial hematocrit ranges. Exchange transfusions performed with whole blood (or, more commonly, packed cells reconstituted to the volume and hematocrit of whole blood using saline or other diluents) are more efficient than those using packed cells alone. They may reduce the number of units of replacement blood products needed but take slightly more time to administer. In adults, blood can be removed from the patient in 500 mL aliquots, followed by infusion of 500 mL of reconstituted blood; this may be repeated for 6-8 units of transfusion. For example, the following technique can be used:

1. Bleed one unit (500 mL) of blood from the patient, infuse 500 mL of saline.

2. Bleed a second unit from the patient, infuse two units of blood.

3. Repeat steps 1 and 2; if the patient has a large red blood cell mass, repeat once more.

In some cases of exchange transfusion, the entire blood or plasma volume is not replaced.

### BRIEF DESCRIPTION OF THE INVENTION

A method and apparatus have been developed for replacement of renal function by periodically removing a substantial volume of patient's blood and simultaneously replacing it with the reconstituted blood. Reconstituted blood consists of the patient's own condensed blood cells and proteins diluted with the sterile physiologic solution. As a result, small molecules and excess water are periodically removed and discarded. Blood cells and proteins are safely stored to be used at the next therapy session.

Clinicians and patients alike recognize the limitations of traditional, three times per week, in-center therapy. Home-based and more frequent therapies offer many potential benefits, including the promise of a more normal lifestyle. Making daily and home therapies a practical reality, however, demands a new approach to RRT other than dialysis. To better address the needs of ESRD patients and society and to eliminate significant limitations of traditional periodic hemodialysis, the inventors have developed a novel method and device that enables RRT that is better, can be performed more frequently, at low cost, faster and may not require a high blood flow AV shunt.

Breaking with the established tradition of periodically circulating high volume of patient's blood through the dialysis filter over 4 hours, that was the paradigm of RRT since its invention, authors propose periodically removing a substantial volume of patient's “toxic” blood and simultaneously (in real time) replacing it with reconstituted “clean” blood. In the disclosed embodiment, the reconstituted blood consists of the patient's own condensed blood cells and proteins diluted with a sterile physiologic replacement solution. As a result, small molecules (solute) and excess water are periodically removed and discarded. Blood cells and proteins are stored (using standard blood component storage methods including refrigeration or freezing) for return during the next therapy session that can be the next day. Each “blood replacement” session is expected to take only 30 to 60 minutes compared the dialysis session of 4 hours. This reduction of time can enable the much desired frequent dialysis without building and staffing more centers, added cost and burden on patient's everyday lives.

Blood flow of approximately 100 to 200 ml/min is required for the blood replacement RRT, compared to the dialysis blood flow of 300 to 400 ml/min. This lower requirement for blood flow results in the additional benefit of the ability to use simplified blood access methods and may allow elimination of AV fistulas and/or shunts that are costly and are poorly tolerated by some patients. The proposed therapy does not rely on diffusion of molecules into the dialysate solution across the filter membrane and is therefore expected to remove middle size molecules as efficiently as small ones. This improved clearance of small and middle molecules is expected to significantly improve the well being of patients. The proposed therapy only requires approximately 2 to 5 liters of sterile replacement solution compared to at least 20 to 150 liters of sterile dialysate used during one traditional dialysis session. Patients undergoing the proposed therapy should be less likely to experience hypotension from inconsistent rates of fluid removal or the acetate used in the dialysate solution and complications of bleeding from blood access.

A proposed embodiment of the therapy can be described as a sequence of following steps.

A. In preparation for initiation of therapy, the patient periodically donates safe amounts of blood. Small amounts of donated blood are stored frozen until the required amount, such as for example 5 liters, is accumulated. There is no need to store the whole blood. Blood cells and proteins can be separated using existing methods and stored. The plasma water can be discarded.

B. Blood access is established. Blood access for the proposed therapy can be a subcutaneous infusion port such as used for drug infusion. A transcutaneous catheter is another form of acceptable blood access. Of course, a graft can be placed in an artery simply to allow a site for repeated vascular access. However, in contrast to the existing graft access, it does not need to be placed between an artery and vein creating an AV fistula. The purpose of the graft is to allow multiple punctures into an arterial blood space that are easy to find, do not bleed and are less painful. A peripheral artery, such as a radial artery, can provide necessary blood flow supply of 100 to 300 ml/min for withdrawal of blood.

C. Patient receives a blood replacement session. The session can take place at a specialty clinic. A substantial amount of whole blood approximating the patient's total intravascular volume, for example 5 liters, is removed from the patient's vein into a blood storage vessel such as a plastic bag. Simultaneously with blood withdrawal, a calculated and controlled infusion of reconstituted blood takes place. It is expected that ESRD patients build up fluid between sessions from drinking and eating. As a result of the difference between the removed and the infused volumes the prescribed net amount of water, such as for example 500 ml to 2 liters, is eliminated from the patient's body to restore the volume balance. Thus, it is expected that the reconstituted blood is infused at a predictably lower rate than the blood withdrawal rate to compensate for this difference. Patients can be weighed before each session to determine how much “net” fluid needs to be removed. The measured excess weight over the patient's dry weight can then be a prescribed goal for the therapy session.

The reconstituted blood is composed of the stored condensed blood cells and proteins previously donated by the same patient and the fresh sterile replacement solution. The replacement solution can be manufactured by a pharmacy or nurse to contain purified water with the addition of essential electrolytes and nutrients following the doctor's prescription for the patient. It is expected that the blood cells and proteins can be diluted by the ratio between 1:2 and 1:4. Normal whole blood consists of 30 to 50% of cells and proteins and 70 to 50% of plasma water. Patients are expected to receive substantially all the blood cells and proteins removed during the previous session. The total volume of the infused replacement solution is adjusted to achieve the desired net fluid loss. Blood can be reconstituted prior to the infusion or prepared during the therapy on-line by mixing stored blood cells and replacement fluid as the mixture is being infused. An anti-coagulant may be given to the patient intravenously and added to collected blood in view of the temporary storage of blood in the bags.

D. Blood withdrawn from the patient during the session can be processed immediately or later. The processing of blood involves separation of blood cells and proteins from plasma water. Toxins and products of metabolism are dissolved in the separated plasma water. This “dirty” plasma water is discarded to be replaced with the “clean” replacement solution during the next therapy session. There are known methods of separating blood cells and proteins from plasma water. Most common are filtration and centrifuging. Large body of engineering knowledge exists and can be applied to adapt these processes for the proposed therapy. The separated blood cells and proteins are then stored for the next session. Several well known methods can be used to preserve blood cells including refrigeration and/or freezing of blood products.

The process described above is somewhat similar to an existing clinical therapy called an exchange transfusion. Similar to our proposed method, exchange transfusions are a potentially life-saving procedure performed by the incremental removal and replacement of the patient's blood to counteract the effects of serious jaundice or changes in the blood (from, for example, sickle cell anemia). However, and in marked contrast to our proposed method and embodiments, exchange transfusions replace the patient's removed blood use blood or plasma from a different donor or set of donors, not the same patient's own blood cells and plasma proteins as is the basis of the replacement fluid.

### BRIEF DESCRIPTION OF THE DRAWINGS

A preferred embodiment and best mode of the invention is illustrated in the attached drawings that are described as follows:

FIG. 1 is a simplified diagram illustrating treatment of an ESRD patient with the present invention.

FIG. 2 illustrates the separation of blood step of the process.

FIG. 3 is a simplified diagram of an embodiment of the Control System used by the invention.

FIG. 4 illustrates software algorithm for blood exchange.

FIG. 5 illustrate method or procedure of exchange dialysis.

### DETAILED DESCRIPTION OF THE INVENTION

For the proposed clinical use, the capability of the invention is to replace renal function by periodically removing substantial volume of patient's blood and replacing it simultaneously with the reconstituted blood. The reconstituted blood contains patient's blood cells and proteins dissolved in the sterile physiologic solution that contains water and vital electrolytes. Small solute molecules and excess water are removed and discarded with the plasma water.

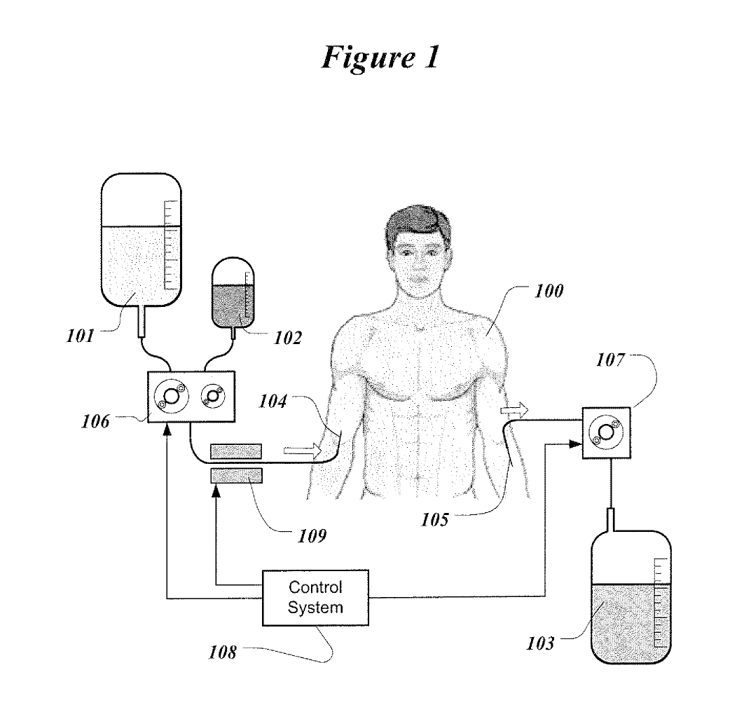

FIG. 1 illustrates the treatment of an ESRD patient with the present invention. Patient 100 can undergo treatment, for example, every day or every second day or four times a week. Treatment can be performed in a clinic or doctor's office. Potentially, home therapy can be envisioned for patients who desire to do so. Therapy requires access to the patient's venous blood. In this illustration, blood access is established with two needles. Infusion needle 104 and phlebotomy needle 105 are inserted into two separate veins (not shown) in patient's arms. It is appreciated that many different forms of blood access such as blood vessel grafts or subcutaneous infusion ports can be used to access blood. Patient's blood is collected in the blood bag 103. This blood contains excess water and various toxins normally removed by kidney such as urea, ammonia and many other small and medium size molecules commonly called solutes. Phlebotomy is shown assisted by a pump 107 to decrease the required time and obtain better control over the procedure. It is appreciated that phlebotomy can be performed by gravity drainage of venous blood.

Blood access needle 104 is used to infuse reconstituted blood. Reconstituted blood is produced by mixing patient's own condensed (often called packed) cells such as red and white blood cells and proteins such as albumin with a replacement solution prepared by pharmacy. In the displayed embodiment, the fluid from the bag with condensed cells and proteins 102 is diluted with sterile solution 101 that consist primarily of distilled water and electrolytes such as sodium and potassium. Electrolytes have substantially the same concentration as in normal human plasma water. For example, replacement solution 101 can contain the following: sodium 140 mEq/L, chloride 120 mEq/L, bicarbonate 25 mEq/L, calcium 2.6 mEq/L, magnesium 1.6 mEq/L, dextrose 124 mg/dl. Other electrolytes, such as small amounts of potassium, can be added to the solution as mandated by the patient's condition and physiological needs.

Blood can be withdrawn from the patient through an 18 Gage or similar withdrawal needle. The needle can be inserted into a suitable peripheral vein in the patient's arm. Blood flow is controlled by the roller pump 107. Before entering the pump, blood passes through one to several meters of plastic tubing. Tubing can be made out of medical PVC of the kind used for IV lines and has internal diameter (ID) of 2.5 to 3 mm. The pump is rotated by a DC motor under microprocessor control. The pump segment (compressed by the rollers) of the tubing has the same ID as the rest of the blood circuit. The system can be designed so that approximately 1 mL of blood is pumped per each full rotation of the pump, e.g. pump speed of 60 RPM corresponds to 60 mL/min. To facilitate repeatable access to blood without risking damage to veins and to have uninterrupted reliable blood flow, the patient can have one or more implanted subcutaneous ports.

A subcutaneous port is also known as a: port-a-cath, port, PAC, or mediport. It is commonly referred to as a “port”. The port is a small metal chamber with a silicone rubber top and an attached catheter. The metal chamber and catheter are placed under the skin. The catheter is then placed in a large vein near the collarbone, in the right atrium of the heart or elsewhere in the venous system. The tip of the catheter sits just inside any large venous structure in or near the right side of the heart. Whenever the port is needed for a blood draw or infusion of medicine or fluids, a special needle called a “Huber needle” is inserted through the skin and into the rubber top of the port. Insertion of the needle into to the port is called accessing. A nurse will clean the skin over the port before accessing the port-a-cath. A port may be placed when a patient needs frequent IV medicine, fluids, or nutrition over a long period of time. The port can also be used to draw blood samples. Ports of different design are available and can be adapted for the use with the invention. Previous attempts to use ports for dialysis met with limited success because high blood flow used by conventional dialysis requires large (14 Gage or 15 Gage needles). These large needles do a lot of damage to the port, especially the septum, and the patient's skin. The inventors believe that the lower blood flow requirements for blood exchange dialysis will enable use of smaller needles (such as 16 Gage, most likely 18 Gage or even 19 Gage and 20 Gage) for access and make ports practical and potentially eliminating need for fistulas and AV grafts.

In the disclosed embodiment, the mixing apparatus 106 contains two pumps. One is for infusion of the condensed cells and proteins 102 and the other for infusion of the replacement solution 101. Controlling the speed of pumps allows reconstitution of blood with the desired hematocrit (dilution) fraction. It is well appreciated that alternate embodiments can also be used where the infused blood is reconstituted manually or automatically before infusion by mixing it with a replacement solution thus eliminating the need for on-line mixing. In this case, a one pump system could be used for infusion.

At the same time, it is essential that the blood removal flow through access 105 and infusion flow through access 104 is substantially synchronized and balanced. Small misbalances between the infusion and withdrawal can be tolerated. If these flows become substantially different at any time, patient can become fluid overloaded and hypertensive or dehydrated and hypotensive. Since patients with ESRD accumulate access water between treatments, it is anticipated that total volume of whole blood removed will be in most cases slightly higher than the total infused volume of reconstituted blood. In the illustrated embodiment pumps 107 and 106 are synchronized and controlled by the programmable electronic Control System 108 (See FIG. 8). The control system 108 integrates embedded software. Basic algorithm controlling the device can be for example Equation 1 below:

Vrs=Vb−Vc−Vfl (Equation 1)

Where Vrs is the amount of replacement solution 101 infused into the patient, Vb is the amount of blood 103 removed from the patient, Vc is the amount of condensed cells and proteins 102 returned to the patient and Vfl is the desired fluid loss. For example, for an average size person, these parameters can be illustrated by the table below. Parameters such as Vb, Vc and Vfl can be set by the operator. The controller software then determines the pump flows needed to implement the desired therapy. For example, if total duration of the therapy session is 30 minutes, average fluid flows can be as illustrated by numbers in the right column of the table I below.

TABLE I

| | | Volume (mL) | Flow (mL/min) |
|---|---|---|---|
| Blood | Vb | 5,000 | 167 |
| Cells and Proteins | Vc | 2,000 | 67 |
| Fluid Loss | Vfl | 500 | 17 |
| Replacement Solution | Vrs | 2,500 | 83 |

The same or similar equation can be used to make a table for the situation in which the cells, protein and replacement solution are reconstituted off-line or prior to infusion that would only require the use of a single replacement pump.

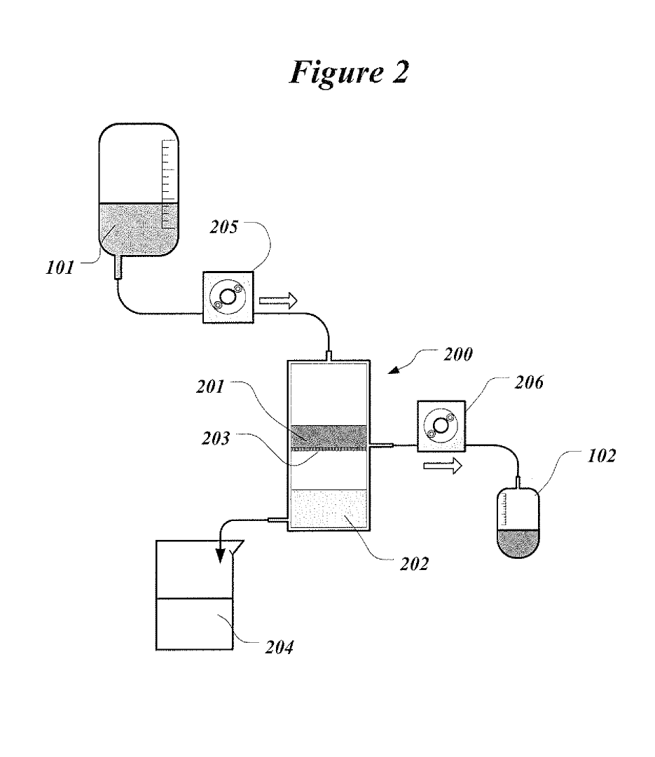

The blood exchange apparatus can be equipped with the safety features needed for rapid infusion of blood such as an air detector (See FIG. 2), pressure sensors and a blood warmer 109 to warm the infused blood to a temperature close to the body temperature of the patient 100.

FIG. 2 illustrates the blood processing step of the invention. Blood 101 containing toxic solute dissolved in plasma water is in the container that can be stored at the processing facility. The blood can be processed within hours of the procedure or later, for example, at night. Blood can be brought from a storage area, such as refrigerator or freezer, for processing. Since processing occurs off-line and the patient not required to be present, blood processing can be automated to reduce labor costs. In the illustrated embodiment, blood 101 is pumped by the pump 205 into the blood separator 200. In this embodiment, the separator is a filter. It is understood that it can also be a centrifuge or other separation methods known in the art. The separator 200 is equipped with a porous membrane 203 that allows passage of water and small solute (plasma water) 202 but retains blood cells and proteins 201. Filtration can be by gravity or assisted by pump pressure. Plasma water 202 is collected in the container 204 and discarded. Condensed cells and proteins 201 are transported to the storage bag 102 by the pump 206 and can be stored until the next blood replacement session.

Across the filter membrane 203 the ultrafiltration occurs. It is appreciated that various suitable blood filtration devices exist that can be adapted for use in the invention. Filter membrane can be made of polymers or cellulose in the form of flat sheets, tubular fibers or spiral wound sheets and cylinders. The flat sheet membrane is shown for simplicity. Regardless of the design, filter membranes are made of a porous material. The pores are permeable to water and small solutes but impermeable to red blood cells, proteins and other blood components that are typically larger than 50,000-60,000 Daltons.

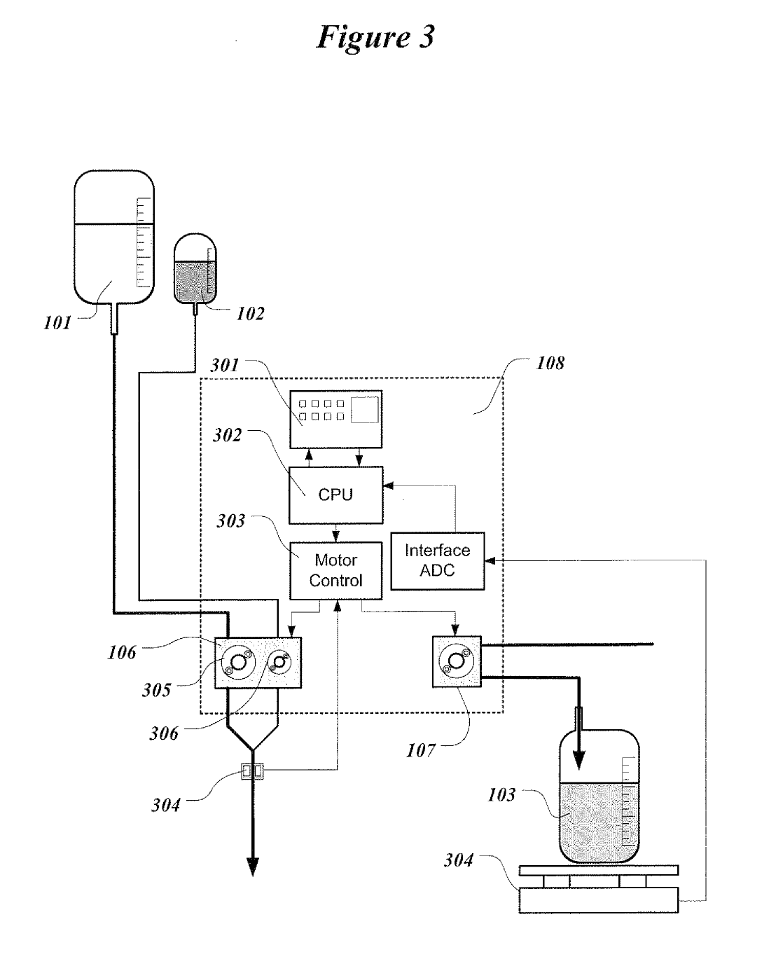

FIG. 3 illustrates the schematic of the Control System. The purpose of the Control System is to ensure that the replacement of blood occurs rapidly in controllable fashion, the prescribed net fluid is removed while the patient is protected from hypotension, acute fluid overload and air embolism. During operation, the present invention requires minimal intervention from user. User sets the maximum rate at which fluid is to be removed from the patient using the control panel 301 that is a part of the Control system 108. Whole blood is withdrawn from the patient via pump 107 into a collection bag 103 that is placed on an electronic scale 304. The purpose of the scale 304 is to provide precise information about the amount of the withdrawn blood for the Control System CPU 302. Software embedded in the CPU 302 calculates the infusion rate of reconstituted blood and communicates it to the Motor Controller 303. The motor controller operates pumps 305, 306 and 107. Air detector 304 is used to stop the infusion and withdrawal pumps in the event of detection of dangerous amount of air in the infusion line. The air detector 304 can be of ultrasonic type and can detect air in amounts exceeding approximately 50 microliters. The detector 304 uses technology based on the difference of the speed of sound in liquid and in gaseous media. If an air bubble is detected, the infusion pumps 305 and 306 is stopped almost instantaneously (within few milliseconds). It is understood that blood can be reconstituted using Equation 1 before the infusion into the patient. In that case, only one infusion pump is needed since blood cells and proteins and replacement solution are already mixed in the required proportion to ensure the desired fluid loss.

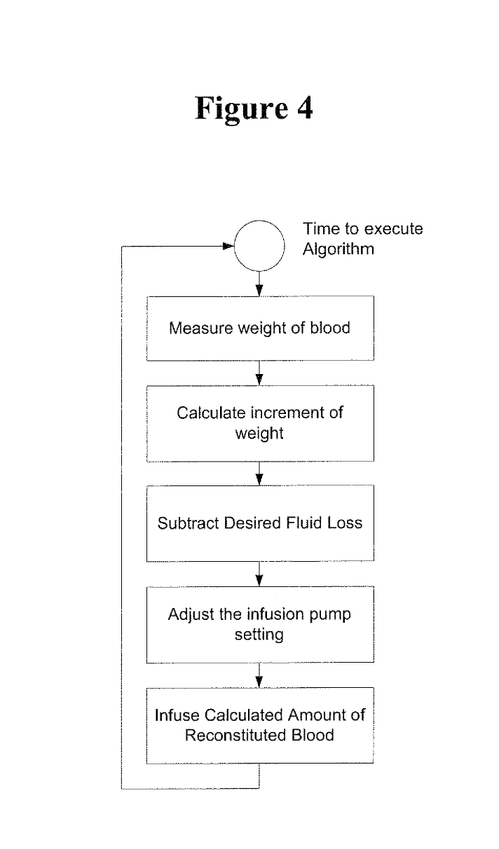

FIG. 4 illustrates a possible algorithm of software that can be embedded in the Control System 108 CPU 302 (See FIGS. 1 and 3). The algorithm is executed in real time by controlling pumps to achieve gradual, synchronized blood replacement with programmed fluid loss without the risk of hypotension or acute fluid overload for the patient. It is expected that gradual loss of fluid at the rate of less than 1.0 liter per hour or 16.6 ml per minute can be tolerated by patients. Vascular volume loss at this rate is be replaced by fluid refilled from other extracellular and intracellular body compartments.

Table II below illustrates one possible therapy session scenario. The patient's initial hematocrit—in this example—is 35% that can be expected for a dialysis patient, who normally have low hematocrits. The patient also desires to lose a total of 1 liter of fluid over the 60 minute therapy session. The software is configured to withdraw 5,000 ml of blood over 60 minutes. The reconstituted blood is made of 1,750 ml of cells and proteins (known to the operator from patient's previous visit) and 2,250 ml of sterile replacement solution made by pharmacy. The resulting reconstituted blood has hematocrit of 44% which is acceptable and not too viscous to be able to infuse via existing classes of infusion pumps and access devices. In general, a hematocrit of the reconstituted blood of<50% is acceptable for infusion. In reality, there is some mixing of blood during the procedure and even higher calculated values can be acceptable. During the procedure, the blood withdrawal pump is controlled at 83 ml/min rate and blood infusion pump at 67 ml/min. Thus, the patient loses fluid at the acceptable rate of 17 ml/minute.

| Session Duration | 60 min | | |
|---|---|---|---|
| Initial Blood Hematocrit | 35% | | |
| | | | Flow |
| | | Volume (mL) | (mL/min) |
| Whole Blood Removed | | 5,000 | 83 |
| Cells and Proteins | | 1,750 | |
| Desired Fluid Loss | | 1,000 | 17 |
| Replacement Solution | | 2,250 | |
| Reconstituted Blood | | 4,000 | 67 |
| Volume | | | |
| Replacement Blood | 44% | | |
| Hematocrit | | | |

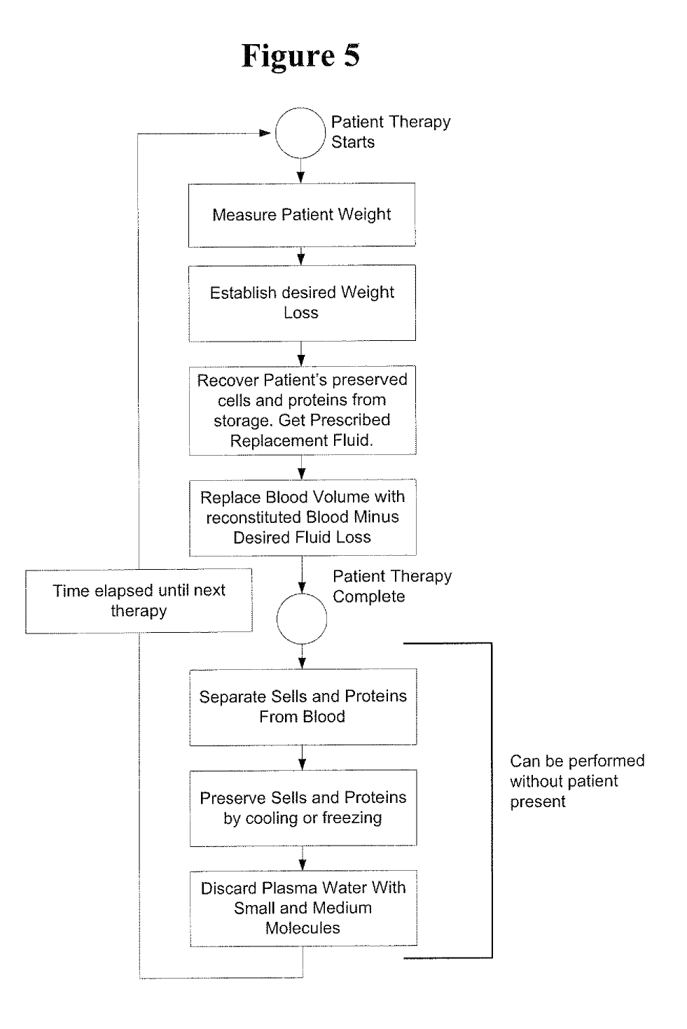

FIG. 5 illustrates one possible scenario for the overall therapy. At the time of therapy, the patient's weight is compared to the desired dry weight and the desired weight loss is calculated. Depending on the desired weight loss, the total therapy time can range from 30 to 90 minutes with 60 minutes likely sufficient to satisfy most clinical cases. The patients preserved cells and proteins are recovered from storage. Based on the amount of cells and desired weight loss, two parameters are calculated: a) time of therapy and b) amount of replacement solution required to add to make up the fully reconstituted blood solution for replacement. The time of therapy is determined by the ability of the patient to tolerate fluid loss without hypotension. Generally, most patients should be able to tolerate 10 to 17 ml/minute fluid loss developing without clinically significant problems. In some cases, a slower fluid removal may be desired. The amount of replacement fluid is determined by the desired fluid loss as illustrated by Equation 1. It is limited by the hematocrit of the reconstituted blood. It is expected that hematocrit of 40-50% is acceptable for replacement infusion. While possible to infuse blood with the hematocrit of 60%, it is rather viscous and may present risks to the patient or equipment.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method for replacement of renal function in patients with renal disease comprising:
a. removing blood from the patient;
b. replacing the removed blood with simultaneous infusion of reconstituted blood, and
c. wherein a flow of the removed blood exceeds a flow of reconstituted removed blood by a predetermined amount corresponding to a desired fluid loss.

2. The method as in claim 1 wherein the blood is removed at the rate of 50 to 100 ml/minute and the desired fluid loss is at the rate of 10 to 20 ml/minute.

3. The method as in claim 1 wherein the reconstituted blood comprises preserved blood cells and proteins of the patient, and a sterile replacement solution.

4. The method as in claim 3 further comprising determining an amount of the sterile replacement solution to achieve the desired fluid loss.

5. The method as in claim 1 further comprising periodically repeating steps a to c on a predefined schedule.

6. The method as in claim 1 further comprising further comprising extracting blood cells and proteins from blood extracted from the patient, storing the extracted blood cells and proteins and including the using the stored extracted blood cells and proteins to form the reconstituted blood replaced in the patient in step b.

7. A method for generating reconstituted blood and infusing the reconstituted blood in a mammalian patient, the method comprising:
a. removing blood from the patient at a removal flow rate;
b. at substantially the same time as the blood is removed, infusing reconstituted blood into the patient at an infusion flow rate, and
c. wherein the removal flow rate exceeds the infusion flow rate.

8. The method of claim 7 wherein the removal flow rate exceeds the infusion flow rate a predetermined flow rate difference.

9. The method as in claim 8 wherein the predetermined flow rate difference is in a range of 10 to 20 ml/minute.

10. The method of claim 7 wherein the removal flow rate exceeds the infusion flow rate for a sufficient period to achieve a predetermined fluid loss in the patient.

11. The method as in claim 7 wherein the removal flow rate is in a range of 50 to 100 ml/minute.

12. The method as in claim 7 wherein the reconstituted blood comprises preserved blood cells and proteins of the patient, and a sterile replacement solution.

13. The method as in claim 12 further comprising determining an amount of the sterile replacement solution to achieve a predetermined desired fluid loss in the patient.

14. The method as in claim 7 further comprising periodically repeating steps a to c on a predefined schedule.

15. The method as in claim 7 wherein the infusion in step b is entirely an infusion of the reconstituted blood.

16. The method as in claim 7 wherein the infusion in step b is a majority of the reconstituted blood.

17. The method as in claim 7 wherein steps a to c are applied to treat a renal condition in the patient.

18. The method as in claim 7 wherein steps a to c are applied as a treatment to replace a natural renal function of the patient.
